(19) United States
(12) Patent Application Publication
(10) Pub. No.: US 2026/0308021 A1
(43) Pub. Date: Oct. 8, 2026

(54) STERILIZING APPARATUS

(71) Applicant: SEOUL VISOYS CO., LTD., Ansan-si (KR)

(72) Inventors: JaeHak JEONG, Ansan-si (KR); Zhisheng Zhang, Shenzhen (CN); MingJun Zhang, Shenzhen (CN)

(21) Appl. No.: 19/639,309

(22) Filed: Apr. 6, 2026

Related U.S. Application Data
(60) Provisional application No. 63/785,565, filed on Apr. 8, 2025.

(51) Int. Cl. C02F 1/32; A61L 2/084; A61L 2/10; C02F 1/30

(52) U.S. Cl. CPC C02F 1/325; A61L 2/084; A61L 2/10; C02F 1/30; A61L 2202/11; A61L 2202/121; A61L 2202/122; A61L 2202/123; A61L 2202/14; C02F 2201/3222; C02F 2201/3228; C02F 2201/326; C02F 2303/04; C02F 2307/12

(57) ABSTRACT

A sterilizing apparatus includes: a receptor including an accommodation space accommodating water; an inlet including a passage for introducing the water and extending in one direction; an outlet including a passage for discharging the water; a light-emitting device generating light for sterilizing the water in the accommodation space; and a plurality of guide ribs protruding upward from a lower surface of the receptor and extending from one side of the receptor to the other side opposite to the one side of the receptor to guide a flow direction of the water in the accommodation space, in which the plurality of guide ribs are arranged such that a separation distance therebetween increases as the plurality of guide ribs extend.

### CROSS REFERENCE TO RELATED APPLICATION

This application claims priority from and the benefit of United States Provisional Patent Application No. 63/785,565, filed on Apr. 8, 2025, which is hereby incorporated by reference for all purposes as if fully set forth herein.

### BACKGROUND

### FIELD

Embodiments of the invention relate to a sterilizing apparatus.

### DISCUSSION OF THE BACKGROUND

A sterilizing apparatus may sterilize an object. Such a sterilizing apparatus may include a light-emitting diode (LED) to sterilize an object. The light-emitting diode can convert an electrical signal into a form of light such as infrared rays, visible rays, and ultraviolet rays using characteristics of a compound semiconductor.

As the light efficiency of light-emitting diodes increases, sterilizing apparatuses are being applied to devices such as dishwashers, water purifiers, and dehumidifiers.

The above information disclosed in this Background section is only for understanding of the background of the inventive concepts, and, therefore, it may contain information that does not constitute prior art.

### SUMMARY

Embodiments of the invention provide a sterilization apparatus including a light-emitting device capable of sterilizing water.

Embodiments of the invention provide a sterilization apparatus having improved sterilization efficiency by increasing light extraction efficiency.

Embodiments of the invention provide a sterilization apparatus in which a moisture permeation path is lengthened to delay moisture permeation and thereby improve reliability.

Additional features of the inventive concepts will be set forth in the description which follows, and in part will be apparent from the description, or may be learned by practice of the inventive concepts.

According to an embodiment of the invention, a sterilizing apparatus includes a receptor having an accommodation space configured to accommodate water, an inlet having a passage for introducing the water and extending in one direction, an outlet having a passage for discharging the water, a light-emitting device configured to generate light for sterilizing the water in the accommodation space, and a plurality of guide ribs protruding upward from a lower surface of the receptor and extending from one side of the receptor to another side, opposite to the one side of the receptor to guide a flow direction of the water in the accommodation space, in which a separation distance between the plurality of guide ribs increases as the plurality of guide ribs extend.

The plurality of guide ribs may be disposed between the inlet and the outlet when viewed in the one direction.

The plurality of guide ribs may be arranged to be inclined at a different angle with respect to the one direction.

The plurality of guide ribs may include a first guide rib disposed closer to the inlet than to the outlet, and a second guide rib disposed closer to the outlet than to the inlet. A height of at least a portion of the first guide rib may increase toward the other side of the receptor, and a height of at least a portion of the second guide rib may decrease toward the other side of the receptor.

The first guide rib may include a first rib region connected to the one side of the receptor, and a second rib region connected to the other side of the receptor and located higher than the first rib region such that the water may flow toward an upper side of the first rib region.

The second guide rib may include a third rib region connected to the one side of the receptor, and a fourth rib region connected to the other side of the receptor. A height of the third rib region may be greater than a height of the fourth rib region such that the water passing through the first rib region may flow further to an upper side of the fourth rib region than to an upper side of the third rib region.

A maximum height of the second rib region and a maximum height of the third rib region may be different from each other.

The sterilizing apparatus may further include an auxiliary rib disposed at a corner of the other side of the receptor and arranged such that a separation distance from a first guide rib of the plurality of guide ribs may increase as one surface of the auxiliary rib extends toward the one side of the receptor, in which the one surface of the auxiliary rib is inclined such that an upper end thereof is disposed closer to the corner of the other side of the receptor than a lower end thereof.

According to another embodiment, a sterilizing apparatus further includes a receptor providing an accommodation space for accommodating water, an inlet providing a passage through which the water is introduced, an outlet providing a passage through which the water is discharged, a light-emitting device configured to generate light for sterilizing the water in the accommodation space, a reflector disposed on an upper side of the receptor to reflect the light generated from the light-emitting device toward the accommodation space, and a plurality of windows formed in the reflector and configured to transmit the light therethrough, in which the light-emitting device is disposed inside each of the plurality of windows.

Some of the plurality of windows may have different areas.

The reflector may include a reflector body having an opening formed inside, and a plurality of separation walls extending in one direction inside the reflector body and arranged to be spaced apart from each other in another direction perpendicular to the one direction to partition the opening into the plurality of windows.

A width of each of the plurality of separation walls may decrease toward the receptor.

The side surfaces of the plurality of separation walls and an inner side surface of the reflector body may be inclined at a predetermined angle with respect to a vertical direction such that an area of each of the plurality of windows may increase toward the receptor.

The inner side surface of the reflector body may be inclined at a greater angle with respect to the vertical direction than the side surfaces of the plurality of separation walls.

The inner side surface of the reflector body may include a first inner side surface extending in another direction perpendicular to the one direction and connected to the plurality of separation walls, and a second inner side surface extending in the one direction and connected to the first inner side surface. The first inner side surface and the second inner side surface may be inclined at different angles with respect to the vertical direction.

The inner side surface of the reflector body may include the first inner side surface extending in another direction perpendicular to the one direction and connected to the plurality of separation walls, and the second inner side surface extending in the one direction and connected to the first inner side surface. The first inner side surface and the second inner side surface may be connected to form a predetermined first radius of curvature, the first inner side surface and the plurality of separation walls may be connected to form a predetermined second radius of curvature, and the first radius of curvature and the second radius of curvature may be different from each other.

The first radius of curvature may be greater than the second radius of curvature.

The sterilizing apparatus may further include a plurality of guide ribs protruding upward from a lower surface of the receptor to guide a flow direction of the water in the accommodation space, and extending from one side of the receptor to an other side, opposite to the one side of the receptor, in which any one of the plurality of guide ribs is arranged to cross any one of the plurality of windows when viewed from above, and another one of the plurality of guide ribs is arranged to cross another one of the plurality of windows when viewed from above.

According to yet another embodiment, a sterilizing apparatus includes a body assembly configured to accommodate water introduced from an outside, a light-emitting device configured to generate light for sterilizing the water inside the body assembly, a substrate disposed inside the body assembly to support the light-emitting device; and a driving device disposed on the substrate to control the light-emitting device, in which the body assembly includes a receptor providing an accommodation space for accommodating the water, a sterilization body providing an inner space for accommodating the receptor, and a body partition partitioning the inner space of the sterilization body into a receptor accommodation for space accommodating the receptor and a driving device accommodation space for accommodating the driving device.

The sterilizing apparatus may further include a reflector supported by the substrate to be disposed in the receptor accommodation space and configured to reflect the light generated from the light-emitting device toward the accommodation space, and a reflector cover disposed between the receptor and the reflector to cover the accommodation space and to transmit the light of the light-emitting device.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the inventive concepts.

FIG. 1 is a perspective view of a sterilizing apparatus according to an embodiment of the invention.

FIG. 2 is an exploded perspective view of the sterilizing apparatus of FIG. 1.

FIG. 3 is an exploded perspective view of the sterilizing apparatus of FIG. 2 viewed from below.

FIG. 4 is a perspective view of a sterilization body coupled with a first packing of the sterilizing apparatus according to an embodiment of the invention.

FIG. 5 is a plan view of the sterilization body of FIG. 4.

FIG. 6 is a cross-sectional view of the sterilizing apparatus taken along line B-B' of FIG. 1.

FIG. 7 is a cross-sectional view of the sterilizing apparatus taken along line C-C' of FIG. 1.

FIG. 8 is a perspective view illustrating the reflector coupled to the substrate of the sterilizing apparatus according to an embodiment of the invention.

FIG. 9 is a plan view of the reflector of FIG. 10.

FIG. 10 is a cross-sectional view of the sterilizing apparatus taken along line A-A' of FIG. 1.

FIG. 11 is an enlarged view of a portion A of FIG. 10.

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z – axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

As customary in the field, some embodiments are described and illustrated in the accompanying drawings in terms of functional blocks, units, and/or modules. Those skilled in the art will appreciate that these blocks, units, and/or modules are physically implemented by electronic (or optical) circuits, such as logic circuits, discrete components, microprocessors, hard-wired circuits, memory elements, wiring connections, and the like, which may be formed using semiconductor-based fabrication techniques or other manufacturing technologies. In the case of the blocks, units, and/or modules being implemented by microprocessors or other similar hardware, they may be programmed and controlled using software (e.g., microcode) to perform various functions discussed herein and may optionally be driven by firmware and/or software. It is also contemplated that each block, unit, and/or module may be implemented by dedicated hardware, or as a combination of dedicated hardware to perform some functions and a processor (e.g., one or more programmed microprocessors and associated circuitry) to perform other functions. Also, each block, unit, and/or module of some embodiments may be physically separated into two or more interacting and discrete blocks, units, and/or modules without departing from the scope of the inventive concepts. Further, the blocks, units, and/or modules of some embodiments may be physically combined into more complex blocks, units, and/or modules without departing from the scope of the inventive concepts.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

Hereinafter, a sterilizing apparatus 1 according to an embodiment of the invention will be described.

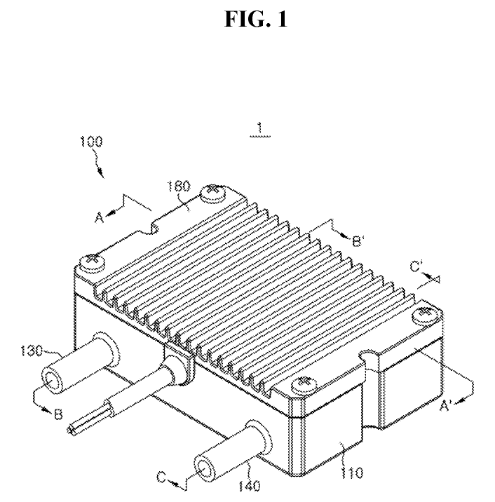

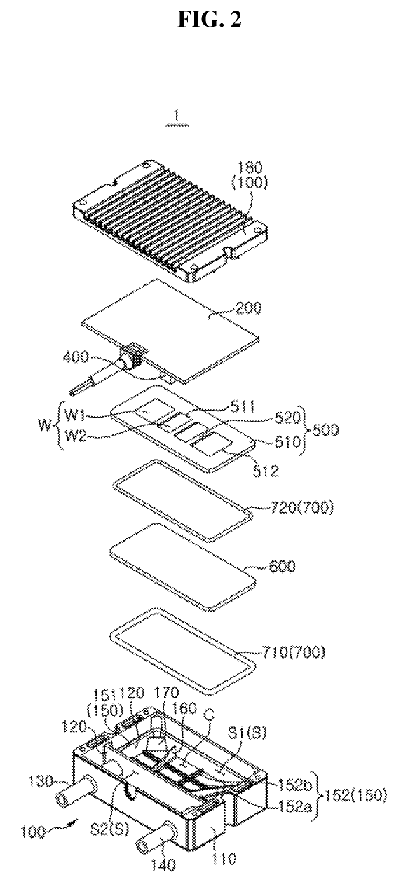

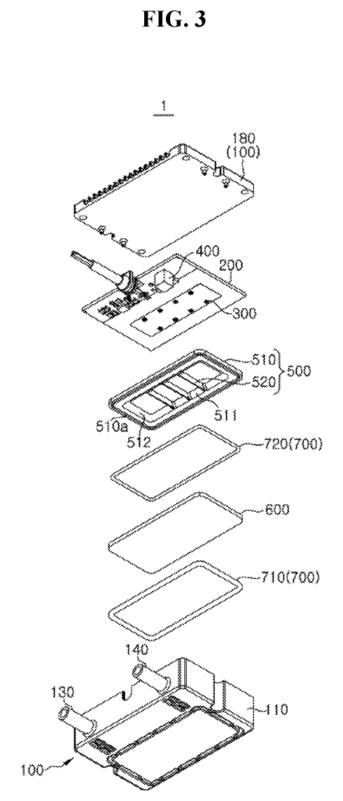

Referring to FIGS. 1 to 3, the sterilizing apparatus 1 according to an embodiment of the invention may sterilize and discharge a sterilization target substance introduced from the outside. The sterilization target substance may be water, but is not limited thereto. The sterilizing apparatus 1 may be mounted on or included in an apparatus using water, such as a water purifier, a dishwasher, or a washing machine, without being limited thereto. For example, the sterilizing apparatus 1 may store and sterilize water used in a dishwasher, and then discharge the water to the dishwasher. According to an embodiment, the sterilizing apparatus 1 may include a body assembly 100, a substrate 200, a light-emitting device 300, a driving device 400, a reflector 500, a reflector cover 600, and a packing 700.

The body assembly 100 may provide an exterior of the sterilizing apparatus 1. The body assembly 100 may accommodate a sterilization target substance introduced from the outside or discharge the accommodated sterilization target substance to the outside. Such a body assembly 100 may include a sterilization body 110, a body partition 120, an inlet 130, an outlet 140, a receptor 150, a guide rib 160, an auxiliary rib 170, and a body cover 180.

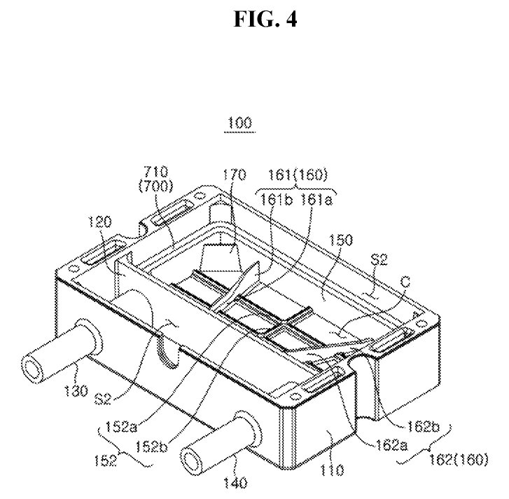

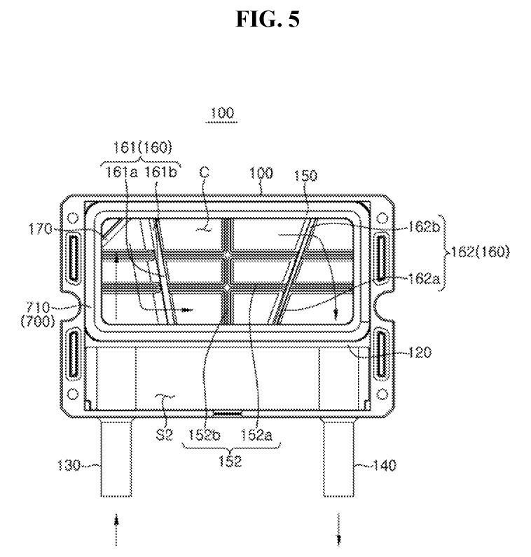

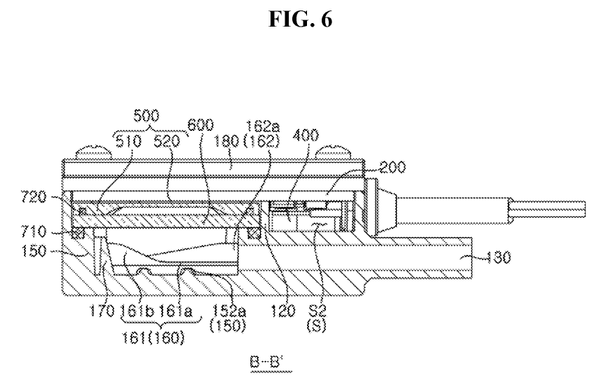

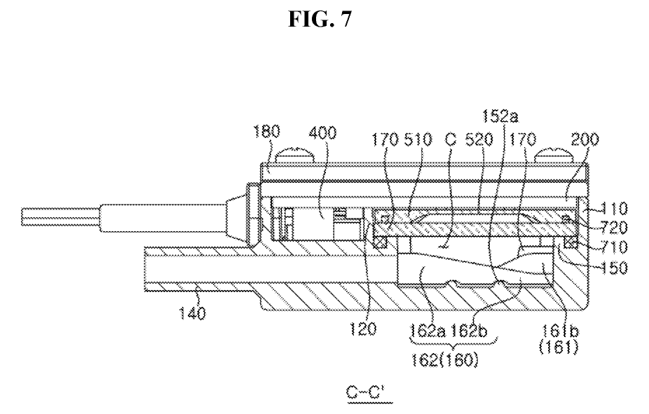

Further referring to FIGS. 4 to 7, the sterilization body 110 may support the inlet 130 and the outlet 140. In addition, the sterilization body 110 may provide an inner space S for accommodating the substrate 200, the light-emitting device 300, the driving device 400, the reflector 500, the reflector cover 600, and the receptor 150.

The body partition 120 may be disposed inside the sterilization body 110 to partition the inner space S of the sterilization body 110 into a receptor accommodation space S1 and a driving device accommodation space S2. The body partition 120 may partition the inner space S such that the driving device accommodation space S2 is located between the receptor accommodation space S1 and the inlet 130. In particular, the body partition 120 may extend in a direction perpendicular to a direction in which the inlet 130 extends to partition the inner space S. The body partition 120 may separate the driving device 400 and the sterilization target substance to prevent the driving device 400 from being polluted by the sterilization target substance.

The inlet 130 may provide a passage through which the sterilization target substance flows such that the sterilization target substance from the outside flows into the receptor 150. The inlet 130 may extend in one direction and pass through the sterilization body 110 and the body partition 120 to be connected to one side of the receptor 150. Accordingly, the sterilization target substance may flow into the receptor 150 through the inlet 130.

The outlet 140 may provide a passage through which the sterilization target substance contained in the receptor 150 flows such that the sterilization target substance of the receptor 150 is discharged to the outside. The outlet 140 may extend in one direction and pass through the sterilization body 110 and the body partition 120 to be connected to one side of the receptor 150. Accordingly, the sterilization target substance may flow from the inside of the receptor 150 to the outside through the outlet 140. The outlet 140 and the inlet 130 may be disposed to be spaced apart from each other in another direction perpendicular to the one direction. Accordingly, a sufficient sterilization time for sterilizing the target substance may be secured.

The receptor 150 may provide an accommodation space C for accommodating the sterilization target substance. The receptor 150 may be disposed in the receptor accommodation space S1. An upper end of the receptor 150 may be disposed below an upper end of the sterilization body 110 and an upper end of the body partition 120. One side of the receptor 150 may be connected to the inlet 130 and the outlet 140. Such a receptor 150 may accommodate the sterilization target substance discharged from the inlet 130 or discharge the sterilization target substance to the outlet 140. In addition, a receptor groove 151 may be formed in the receptor 150.

A first packing 710 to be described later may be inserted into the receptor groove 151. The receptor groove 151 may be recessed downward from an upper end of the receptor 150 and extend along an inner circumferential surface of the receptor 150. Accordingly, the first packing 710 may be stably inserted.

A plurality of guide ribs 160 may be formed and supported by the receptor 150 so as to be disposed in the accommodation space C, thereby guiding a flow direction of water inside the receptor 150. The plurality of guide ribs 160 may extend from one side of the receptor 150 toward the other side opposite to the one side of the receptor 150. Hereinafter, for convenience of description of the embodiment, one side of the receptor 150 will be referred to as a front side, and the other side of the receptor 150 will be referred to as a rear side. In addition, one direction will be referred to as a front and rear direction, and another direction will be referred to as a left and right direction. Such a plurality of guide ribs 160 may guide the water such that the water introduced into the receptor 150 through the inlet 130 flows toward the outlet 140 while flowing in the front and rear direction.

The plurality of guide ribs 160 may be disposed to be spaced apart from each other in the left and right direction. The plurality of guide ribs 160 may be arranged such that a separation distance increases toward the rear side of the receptor 150. Such a plurality of guide ribs 160 may be arranged to be inclined at different angles with respect to the front and rear direction. In addition, the plurality of guide ribs 160 may be disposed between the inlet 130 and the outlet 140 when viewed in the front and rear direction. The plurality of guide ribs 160 may include a first guide rib 161 and a second guide rib 162.

The first guide rib 161 may be disposed closer to the inlet 130 than to the outlet 140. The first guide rib 161 may be disposed closer to a right side of the receptor 150 toward the rear side of the receptor 150. Such a first guide rib 161 may be disposed closer to a corner of a right rear wall surface of the receptor 150, thereby increasing a flow rate of the sterilization target substance at the corner. Accordingly, the sterilization target substance may be guided to move to an entire region of the receptor 150 without being stagnant in the corner region, thereby increasing sterilization efficiency.

In addition, a height of at least a portion of the first guide rib 161 may increase toward the rear side of the receptor 150. Such a first guide rib 161 may include a first rib region 161a and a second rib region 161b.

The first rib region 161a may be connected to the front side of the receptor 150. A height of the first rib region 161a may be lower than a height of the second rib region 161b. The sterilization target substance may pass through a region directly above the first rib region 161a. Accordingly, a vortex of the sterilization target substance may be generated in the receptor 150 to increase sterilization efficiency.

The second rib region 161b may be connected to the rear side of the receptor 150. The second rib region 161b may be higher than the first rib region 161a. Such a second rib region 161b may guide the sterilization target substance such that the sterilization target substance introduced into the receptor 150 flows toward the first rib region 161a. Accordingly, the water in the receptor 150 may flow toward the front side of the receptor 150 by the second rib region 161b and pass through the region directly above the first rib region 161a. A height of the second rib region 161b may be higher than a height of a fourth rib region 162b. Accordingly, a retention time of the sterilization target substance in the receptor 150 may be increased to increase sterilization efficiency.

The second guide rib 162 may be disposed closer to the outlet 140 than to the inlet 130. The second guide rib 162 may be disposed closer to a left side of the receptor 150 toward the rear side of the receptor 150. In addition, a height of at least a portion of the second guide rib 162 may decrease toward the rear side of the receptor 150. The first guide rib 161 and the second guide rib 162 may be inclined at inclinations in opposite directions in height. Accordingly, a time during which the sterilization target substance is retained in a central portion of the accommodation space C may be increased to increase sterilization efficiency. In addition, the first guide rib 161 and the second guide rib 162 may be inclined at inclinations in opposite directions in the left and right direction. More particularly, the first guide rib 161 may be arranged to be inclined toward the wall surface of the receptor 150 to the left rear, and the second guide rib 162 may be arranged to be inclined toward the wall surface of the receptor 150 to the right rear. Accordingly, a time during which the sterilization target substance is retained in the central portion of the accommodation space C may be increased to increase sterilization efficiency. Such a second guide rib 162 may include a third rib region 162a and the fourth rib region 162b.

The third rib region 162a may be connected to the front side of the receptor 150. A height of the third rib region 162a may be greater than the height of the fourth rib region 162b. Such a third rib region 162a may guide the sterilization target substance such that the sterilization target substance passing through the second rib region 161b flows toward the rear side of the receptor 150. The height of the third rib region 162a may be higher than the height of the second rib region 161b. Accordingly, a movement path of the sterilization target substance may be lengthened to increase sterilization efficiency.

The fourth rib region 162b may be connected to the rear side of the receptor 150. The height of the fourth rib region 162b may be lower than the height of the third rib region 162a. The sterilization target substance may pass through a region directly above the fourth rib region 162b. The sterilization target substance guided to the rear side of the receptor 150 by the third rib region 162a may pass through the fourth rib region 162b and flow toward the front side of the receptor 150 to be introduced into the outlet 140. Accordingly, the movement path of the sterilization target substance may be lengthened to increase sterilization efficiency.

Furthermore, an auxiliary guide rib 152 may be formed on the receptor 150.

The auxiliary guide rib 152 may be disposed in the accommodation space C to be disposed between the first guide rib 161 and the second guide rib 162. A height of the auxiliary guide rib 152 may be lower than a minimum height of the second rib region 161b of the first guide rib 161 or a minimum height of the fourth rib region 162b of the second guide rib 162. Such an auxiliary guide rib may increase the retention time of the sterilization target substance to increase sterilization efficiency. In addition, the auxiliary guide rib 152 may include a horizontal auxiliary guide rib 152a and a vertical auxiliary guide rib 152b.

The horizontal auxiliary guide rib 152a may extend in the left and right direction to cross the first guide rib 161 and the second guide rib 162. In particular, the horizontal auxiliary guide rib 152a may pass through the first guide rib 161 and the second guide rib 162. A plurality of horizontal auxiliary guide ribs 152a may be formed and disposed to be spaced apart from each other in the front and rear direction.

The vertical auxiliary guide rib 152b may extend in the front and rear direction and be disposed between the first guide rib 161 and the second guide rib 162. In addition, the vertical auxiliary guide rib 152b may be connected to the plurality of horizontal auxiliary guide ribs 152a.

The auxiliary rib 170 may guide the sterilization target substance such that the sterilization target substance introduced into the receptor 150 flows toward the first guide rib 161. The auxiliary rib 170 may be disposed at a rear corner of the receptor 150. In addition, the auxiliary rib 170 may be arranged such that a separation distance from the first guide rib 161 increases as one surface thereof extends toward the front side of the receptor 150. Both ends of the one surface of the auxiliary rib 170 may be connected to the rear side and the right side of the receptor 150. In addition, the one surface of the auxiliary rib 170 may be inclined such that an upper end thereof is disposed closer to the rear corner of the receptor 150 than a lower end thereof. Accordingly, the sterilization target substance may flow to a region close to the light-emitting device 300, thereby increasing sterilization efficiency. In addition, at least a portion of the auxiliary rib 170 may be disposed to overlap the inlet 130 when viewed in the front and rear direction. The water discharged from the inlet 130 may flow toward the auxiliary rib 170 inside the receptor 150. In addition, the water may flow toward the first guide rib 161 by the auxiliary rib 170. Accordingly, the movement path of the sterilization target substance may be lengthened to increase sterilization efficiency.

In addition, a height of the auxiliary rib 170 may be greater than a height of the first guide rib 161 and a second guide rib 162, but is not limited thereto. In particular, at least a portion of the height of the auxiliary rib 170, the height of the second rib region 161b, and the height of the third rib region 162a may be identical to each other. Accordingly, the sterilization target substance may be mixed inside the receptor 150, thereby increasing sterilization efficiency.

The body cover 180 may be disposed on an upper side of the sterilization body 110 to cover the inner space S of the sterilization body 110. The body cover 180 may be disposed to contact the substrate 200. Such a body cover 180 may dissipate heat transferred from the light-emitting device 300, the driving device 400, and the substrate 200 to the outside. Accordingly, the body cover 180 may function as a heat sink. The body cover 180 may be formed of a metal material having high thermal conductivity, such as aluminum or copper, without being limited thereto. The body cover 180 may be formed of a material having higher thermal conductivity than the sterilization body 110 for efficient heat dissipation. Fins facilitating heat dissipation may be additionally disposed on the body cover 180. In this case, a cross-sectional area of the body cover 180 may be larger than that of the substrate 200.

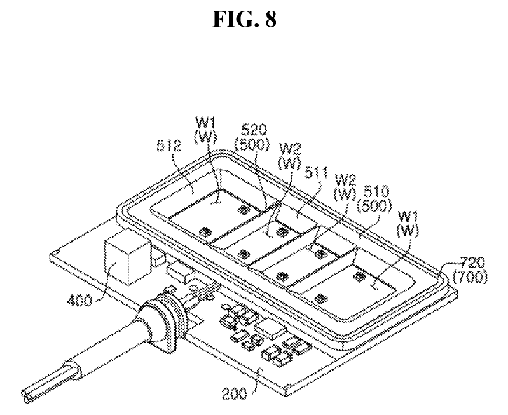

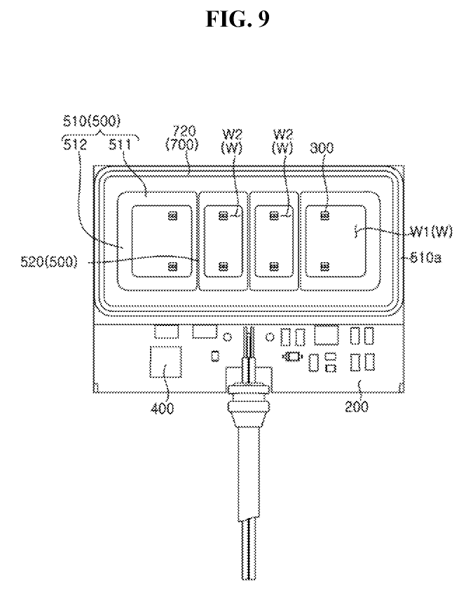

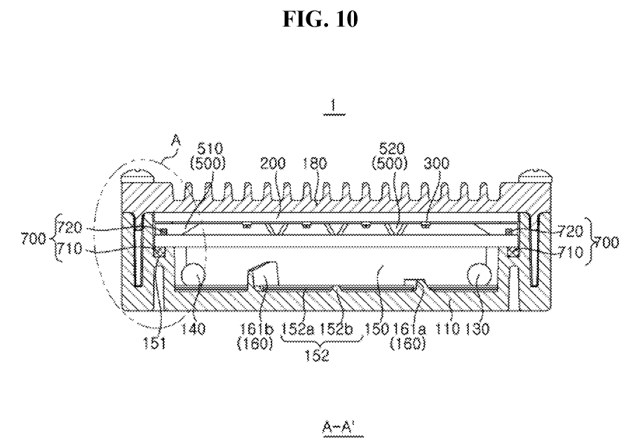

Further, referring to FIGS. 8 to 10, the light-emitting device 300 and the driving device 400 may be disposed on the substrate 200. For example, the substrate 200 may be a PCB (printed circuit board) substrate on which an electric circuit is printed. In addition, the substrate 200 may be a thin-film transistor (TFT) backplane, without being limited thereto. The substrate 200 may be formed of a metal material having thermal conductivity, such as Cu or Al. However, the inventive concepts are not limited thereto, and in some embodiments, the substrate 200 may include one or more insulating materials such as FR1, CEM-1, FR-4, PMMA, PCT, PPA, or the like, thereby preventing a short circuit between circuits. Here, FR1 is a material in which a copper foil and a laminate paper are stacked, and CEM-1 is a material in which a copper foil, a glass fiber fabric, a laminate paper, and a glass fiber fabric are sequentially stacked. In addition, FR-4 is a material in which a copper foil and a glass fiber fabric or a glass fiber fabric are stacked. In addition, the substrate 200 may include a ceramic such as alumina (Al₂O₃), aluminum nitride (AlN), or ZTA (zirconia toughened alumina), without being limited thereto.

Such a substrate 200 may be accommodated in the sterilization body 110. In addition, the substrate 200 may be disposed on an upper side of the body partition 120 such that the light-emitting device 300 is disposed in the receptor accommodation space S1 and the driving device 400 is disposed in the driving device accommodation space S2. Accordingly, interference with the driving device 400 caused by light energy emitted from the light-emitting device 300 may be minimized, thereby increasing stability.

A plurality of light-emitting devices 300 may be formed and disposed on the substrate 200 to generate light for sterilizing or optically treating the sterilization target substance accommodated in the receptor 150. The light-emitting device 300 may be electrically connected to the substrate 200 and receive electricity from the outside through an electric circuit to generate light. For example, the light-emitting device 300 may generate blue, green, red, white light, UV light, or the like. The light-emitting device 300 may be a light-emitting device emitting a wavelength having sterilization power. The light-emitting device 300 may be a device having a plurality of semiconductor layers based on AlₓGa₁₋ₓN (aluminum gallium nitride). The semiconductor layers of the light-emitting device 300 may include a first-type semiconductor layer, a second-type semiconductor layer, and an active layer. The plurality of light-emitting devices 300 may be spaced apart from the driving device 400 to be disposed in the receptor accommodation space S1. The plurality of light-emitting devices 300 may be disposed in a region directly above the receptor 150.

The driving device 400 may be disposed on the substrate 200 and control the light-emitting device 300 such that light is generated from the light-emitting device 300. The driving device 400 may be electrically connected to the substrate 200 and receive electricity from the outside through an electric circuit to control the light-emitting device 300. The driving device 400 may be spaced apart from the light-emitting device 300 to be disposed in the driving device accommodation space S2.

The reflector 500 may reflect the light generated from the plurality of light-emitting devices 300 toward the accommodation space C of the receptor 150. A plurality of windows W may be formed in the reflector 500. The window W formed in the reflector body 510 may be a through-hole. The reflector 500 may be disposed on the substrate 200 such that one or more light-emitting devices 300 may be disposed inside each of the plurality of windows W. Accordingly, the light generated from the plurality of light-emitting devices 300 may pass through the plurality of windows W. The plurality of windows W may be arranged in the left and right direction. At least a portion of the plurality of windows W may be formed to have different sizes. In addition, a size of an area of the plurality of windows W may be formed to increase toward the receptor 150. The plurality of windows W may include a first window W1 and a second window W2.

A plurality of first windows W1 may be formed. The second window W2 may be disposed inside the plurality of first windows W1. The first windows W1 may be disposed outside the second window W2. A size of the first windows W1 may be formed to be greater than a size of the second window W2. An inner area of the first windows W1 may be formed to be larger than an inner area of the second window W2. The inner area of the first windows W1 may be formed to be 1.5 times to 4 times larger than the inner area of the second window W2. Accordingly, the light-emitting device 300 disposed in an area of the first window W1 may have a wider beam angle than the light-emitting device 300 disposed in an area of the second window W2. In this case, the light-emitting device 300 disposed in the area of the first window may sterilize a wide area. When viewed from above, the first guide rib 161 may be arranged to cross any one of the plurality of first windows W1, and the second guide rib 162 may be arranged to cross another one of the plurality of first windows W1. In this case, the first guide rib 161 or the second guide rib 162 may generate a vortex in the sterilization target substance in the accommodation space C, and the first window W1 may sterilize the sterilization target substance in a wide direction, thereby increasing sterilization efficiency.

A plurality of second windows W2 may be formed. The plurality of second windows W2 may be disposed between the plurality of first windows W1. A size of the second windows W2 may be formed to be smaller than the size of the first windows W1. Further, the light-emitting device 300 disposed in an area of the second window W2 may have a narrower beam angle than the light-emitting device 300 disposed in the area of the first window W1 in the accommodation space C. Accordingly, strong light may be irradiated onto a narrow area to increase a sterilization power for the sterilization target substance. Strong light may be irradiated onto a region narrower than a central region having a relatively faster flow rate than an outer side of the accommodation space C, thereby increasing sterilization efficiency.

The reflector 500 may include a reflector body 510 and a plurality of separation walls 520.

The reflector body 510 may be disposed between the receptor 150 and the substrate 200, and at least a portion of the reflector body 510 may extend along an edge portion of the receptor 150 or an edge portion of the substrate 200. An opening may be formed inside the reflector body 510. An inner side surface of the reflector body 510 forming such an opening may be inclined at a predetermined angle with respect to a vertical direction such that an area of each of the plurality of windows W increases toward the receptor 150. In other words, when the reflector body 510 is viewed from below, an upper end of the inner side surface of the reflector body 510 may be disposed further inward than a lower end of the inner side surface of the reflector body 510. In addition, the inner side surface of the reflector body 510 may be inclined at a greater angle with respect to the vertical direction than side surfaces of the plurality of separation walls 520. Accordingly, an emission angle of the light-emitting device 300 may be adjusted to easily adjust a sterilization region. Such a reflector body 510 may include a first inner side surface 511 and a second inner side surface 512.

The first inner side surface 511 may extend in the left and right direction. In addition, the plurality of separation walls 520 may be connected to the first inner side surface 511. The first inner side surface 511 may be inclined at a predetermined first angle with respect to the vertical direction.

The second inner side surface 512 may extend in the front and rear direction to be connected with the first inner side surface 511. The second inner side surface 512 may be inclined at a predetermined second angle with respect to the vertical direction. The second angle may be formed to be larger than the first angle. In particular, the second inner side surface 512 may be inclined at a greater angle with respect to the vertical direction than the first inner side surface 511. In this manner, the beam angle of the light-emitting device 300 may be adjusted to be wider toward the outer side than on an inner side surface, thereby widening the sterilization region. In addition, the second inner side surface 512 and the first inner side surface 511 may be connected to form a predetermined first radius of curvature. Accordingly, light loss may be reduced.

In addition, a body groove 510a may be formed in the reflector body 510. The body groove 510a may be recessed upward from a lower surface of the reflector body 510 and extend along a circumferential surface of the reflector body 510. A second packing 720 to be described later may be inserted into the body groove 510a. Accordingly, moisture penetration may be blocked to increase reliability of the sterilizing apparatus.

The plurality of separation walls 520 may be disposed inside the reflector body 510 to partition the opening into the plurality of windows W. The plurality of separation walls 520 may have the same length in the front and rear direction as the windows W. The plurality of separation walls 520 may be arranged to be spaced apart from each other in the left and right directions. Such a plurality of separation walls 520 may be connected to the first inner side surface 511. Side surfaces of the plurality of separation walls 520 may be inclined at a predetermined third angle with respect to the vertical direction such that an area of each of the plurality of windows W increases toward the receptor 150. The third angle may be smaller than the first angle. Accordingly, the beam angle of the light-emitting device 300 disposed in the central region of the accommodation space C may be narrowed to intensively irradiate light to the central region, thereby increasing sterilization efficiency. A width of such a plurality of separation walls 520 may decrease toward the receptor 150. The width may be a length in the left and right directions. Accordingly, light loss may be reduced while adjusting the beam angle of the light of the light-emitting device 300, thereby increasing light efficiency. The plurality of separation walls 520 and the first inner side surface 511 may be connected to form a predetermined second radius of curvature. The first radius of curvature and the second radius of curvature may be formed differently from each other. In particular, the first radius of curvature may be formed to be larger than the second radius of curvature. Accordingly, the beam angle of the light-emitting device 300 disposed in the central region of the accommodation space C may be narrowed to intensively irradiate light to the central region, thereby increasing sterilization efficiency.

The reflector cover 600 may be disposed between the reflector body 510 and the receptor 150. The reflector cover 600 may cover the plurality of windows W to block the sterilization target substance of the receptor 150 from entering the plurality of windows W. In addition, the reflector cover 600 may be formed such that light generated from the plurality of light-emitting devices 300 may be transmitted therethrough. The reflector cover 600 may be formed of a material having light transmittance, such as quartz, borosilicate glass, or PET, without being limited thereto.

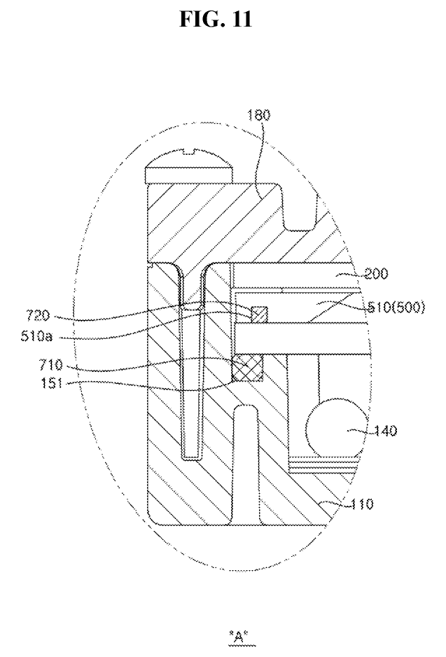

Further referring to FIG. 11, the packing 700 may block water flowing in the receptor 150 from flowing toward the light-emitting device 300 or block external foreign substances from being introduced into the receptor accommodation space S1. The packing 700 may be formed of a material having elasticity, such as rubber or silicone, without being limited thereto. For example, the packing 700 may be an O-ring, without being limited thereo. A plurality of packings 700 may be formed and accommodated inside the sterilization body 110. The plurality of packings 700 may include the first packing 710 and the second packing 720.

The first packing 710 may be inserted into the receptor groove 151. Such a first packing 710 may extend along the receptor groove 151. The first packing 710 may be pressed by a lower surface of the reflector cover 600 while being inserted into the receptor groove 151.

The second packing 720 may be inserted into the body groove 510a. Such a second packing 720 may extend along the body groove 510a. The second packing 720 may be pressed by an upper surface of the reflector cover 600 while being inserted into the body groove 510a. As such, arranging the plurality of packings may increase waterproofness.

Embodiments of the invention may sterilize water accommodated in an accommodation space of a receptor.

Embodiments of the invention may guide a flow direction of water flowing in the accommodation space of the receptor.

Embodiments of the invention may adjust a flow rate inside the receptor to improve a sterilization effect.

Embodiments of the invention may increase light extraction efficiency to increase sterilization efficiency.

Embodiments of the invention may increase a length of a moisture penetration path to delay moisture penetration and improve reliability.

Embodiments of the invention may facilitate heat dissipation to improve reliability.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A sterilizing apparatus comprising:
a receptor including an accommodation space configured to accommodate water;
an inlet including a passage for introducing the water and extending in one direction;
an outlet including a passage for discharging the water;
a light-emitting device configured to generate light for sterilizing the water in the accommodation space; and
a plurality of guide ribs protruding upward from a lower surface of the receptor and extending from one side of the receptor to another side, opposite to the one side of the receptor, to guide a flow direction of the water in the accommodation space,
wherein a separation distance between the plurality of guide ribs increases as the plurality of guide ribs extends.

2. The sterilizing apparatus of claim 1, wherein the plurality of guide ribs is disposed between the inlet and the outlet when viewed in the one direction.

3. The sterilizing apparatus of claim 1, wherein the plurality of guide ribs is arranged to be inclined at a different angle with respect to the one direction.

4. The sterilizing apparatus of claim 1, wherein the plurality of guide ribs comprises:
a first guide rib disposed closer to the inlet than to the outlet; and
a second guide rib disposed closer to the outlet than to the inlet, and
wherein a height of at least a portion of the first guide rib increases toward the other side of the receptor, and a height of at least a portion of the second guide rib decreases toward the other side of the receptor.

5. The sterilizing apparatus of claim 4, wherein the first guide rib comprises:
a first rib region connected to the one side of the receptor; and
a second rib region connected to the other side of the receptor and located higher than the first rib region such that the water flows toward an upper side of the first rib region.

6. The sterilizing apparatus of claim 5, wherein the second guide rib comprises:
a third rib region connected to the one side of the receptor; and
a fourth rib region connected to the other side of the receptor, and
wherein a height of the third rib region is greater than a height of the fourth rib region such that the water passing through the first rib region flows further to an upper side of the fourth rib region than to an upper side of the third rib region.

7. The sterilizing apparatus of claim 6, wherein a maximum height of the second rib region and a maximum height of the third rib region are different from each other.

8. The sterilizing apparatus of claim 3, further comprising:
an auxiliary rib disposed at a corner of the other side of the receptor and arranged such that a separation distance from a first guide rib of the plurality of guide ribs increases as one surface of the auxiliary rib extends toward the one side of the receptor,
wherein the one surface of the auxiliary rib is inclined such that an upper end thereof is disposed closer to the corner of the other side of the receptor than a lower end thereof.

9. A sterilizing apparatus comprising:
a receptor providing an accommodation space for accommodating water;
an inlet providing a passage through which the water is introduced;
an outlet providing a passage through which the water is discharged;
a light-emitting device configured to generate light for sterilizing the water in the accommodation space;
a reflector disposed on an upper side of the receptor to reflect the light generated from the light-emitting device toward the accommodation space; and
a plurality of windows formed in the reflector and configured to transmit the light therethrough,
wherein the light-emitting device is disposed inside each of the plurality of windows.

10. The sterilizing apparatus of claim 9, wherein some of the plurality of windows have different areas.

11. The sterilizing apparatus of claim 9, wherein the reflector comprises:
a reflector body having an opening formed inside; and
a plurality of separation walls extending in one direction inside the reflector body and arranged to be spaced apart from each other in another direction perpendicular to the one direction to partition the opening into the plurality of windows.

12. The sterilizing apparatus of claim 11, wherein a width of each of the plurality of separation walls decreases toward the receptor.

13. The sterilizing apparatus of claim 11, wherein side surfaces of the plurality of separation walls and an inner side surface of the reflector body are inclined at a predetermined angle with respect to a vertical direction such that an area of each of the plurality of windows increases toward the receptor.

14. The sterilizing apparatus of claim 13, wherein the inner side surface of the reflector body is inclined at a greater angle with respect to the vertical direction than the side surfaces of the plurality of separation walls.

15. The sterilizing apparatus of claim 13, wherein the inner side surface of the reflector body comprises:
a first inner side surface extending in another direction perpendicular to the one direction and connected to the plurality of separation walls; and
a second inner side surface extending in the one direction and connected to the first inner side surface, and
wherein the first inner side surface and the second inner side surface are inclined at different angles with respect to the vertical direction.

16. The sterilizing apparatus of claim 15, wherein the inner side surface of the reflector body comprises:
the first inner side surface extending in another direction perpendicular to the one direction and connected to the plurality of separation walls; and
the second inner side surface extending in the one direction and connected to the first inner side surface,
wherein the first inner side surface and the second inner side surface are connected to form a predetermined first radius of curvature,
wherein the first inner side surface and the plurality of separation walls are connected to form a predetermined second radius of curvature, and
wherein the first radius of curvature and the second radius of curvature are different from each other.

17. The sterilizing apparatus of claim 16, wherein the first radius of curvature is greater than the second radius of curvature.

18. The sterilizing apparatus of claim 9, further comprising:
a plurality of guide ribs protruding upward from a lower surface of the receptor to guide a flow direction of the water in the accommodation space, and extending from one side of the receptor to another side, opposite to the one side of the receptor,
wherein any one of the plurality of guide ribs is arranged to cross any one of the plurality of windows when viewed from above, and another one of the plurality of guide ribs is arranged to cross another one of the plurality of windows when viewed from above.

19. A sterilizing apparatus comprising:
a body assembly configured to accommodate water introduced from an outside;
a light-emitting device configured to generate light for sterilizing the water inside the body assembly;
a substrate disposed inside the body assembly to support the light-emitting device; and
a driving device disposed on the substrate to control the light-emitting device,
wherein the body assembly comprises:
a receptor providing an accommodation space for accommodating the water;
a sterilization body providing an inner space for accommodating the receptor; and
a body partition partitioning the inner space of the sterilization body into a receptor accommodation space for accommodating the receptor, and a driving device accommodation space for accommodating the driving device.

20. The sterilizing apparatus of claim 19, further comprising:
a reflector supported by the substrate to be disposed in the receptor accommodation space and configured to reflect the light generated from the light-emitting device toward the accommodation space; and
a reflector cover disposed between the receptor and the reflector to cover the accommodation space and to transmit the light of the light-emitting device.
